(19) United States
(12) Patent Application Publication
(10) Pub. No.: US 2026/0060910 A1
(43) Pub. Date: Mar. 5, 2026

(54) COMPOSITION FOR ACTIVATING EXTERNAL BARRIER

(71) Applicants: Yuki Gosei Kogyo Co., Ltd., Tokyo (JP); Kyushu University, National University Corporation, Fukuoka (JP)

(72) Inventors: Tetsuji NODA, Tokyo (JP); Yoshinori KATAKURA, Fukuoka (JP)

(21) Appl. No.: 19/086,968

(22) Filed: Mar. 21, 2025

(30) Foreign Application Priority Data
Mar. 22, 2024 (JP) 2024-046041

(51) Int. Cl. A61K 8/44; A23L 33/175; A61K 8/14; A61K 8/60; A61K 8/98; A61Q 19/00

(52) U.S. Cl. CPC A61K 8/44; A23L 33/175; A61K 8/14; A61K 8/606; A61K 8/981; A61Q 19/00

(57) ABSTRACT

The object of the present invention is to provide a composition that improves skin barrier function. The problem can be solved by the composition for activating the skin barrier of the present invention, comprising β-alanine or a salt thereof.

### CROSS REFERENCE TO RELATED APPLICATIONS

This application is a U.S. Non-Provisional Application which claims priority from Japanese application number 2024-046041, filed Mar. 22, 2024. The entire contents of this prior application are hereby incorporated herein by reference.

### SEQUENCE LISTING

The instant application contains a Sequence Listing, created on Mar. 17, 2025; the file, in XML format, is designated YGK-121-U.S. Pat. No. 2,352,136 Sequence Listing.xml and is 6,364 bytes in size. The file is hereby incorporated by reference in its entirety into the instant application.

### TECHNICAL FIELD

The present invention relates to a composition for activating the skin barrier. According to the present invention, the skin barrier function of the skin can be enhanced.

### BACKGROUND ART

The epidermis is located on the outermost side of the skin and is responsible for the barrier function of protecting the body from external stimuli. From the top, the epidermis consists of the stratum corneum, stratum granulosum, stratum spinosum, and stratum basale, the majority of which are occupied by keratinocytes, and pigment cells (melanocytes) are present in the stratum basale.

### CITATION LIST

### Patent Literature

[Patent literature 1] JP 2021-511388A

### SUMMARY OF INVENTION

### Technical Problem

The skin barrier is also called the external barrier, and it has been reported that, for example, ambroxol improves the skin barrier function (Patent literature 1). However, a more effective and safe method for improving the skin barrier function has been desired.

The object of the present invention is therefore to provide a composition that improves the skin barrier function.

### Solution to Problem

The present inventors have conducted intensive studies into compositions that improve the skin barrier function, and have surprisingly found that β-alanine improves the skin barrier function.

The present invention is based on this finding.

Accordingly, the present invention relates to:
[1] a composition for activating skin barrier, comprising β-alanine or a salt thereof,
[2] the composition for activating the skin barrier of the item [1], wherein filaggrin, involucrin, or keratin 10 is activated,
[3] a composition for activating the skin barrier, comprising a culture product secreted from muscle cells by β-alanine or a salt thereof,
[4] the composition for activating the skin barrier of the item [3], wherein the culture product is an exosome,
[5] a composition for secreting an exosome having an skin barrier activation effect from muscle cells, comprising β-alanine or a salt thereof,
[6] the composition of the item [4] or [5], wherein miRNA content in the exosome varies with respect to the miRNA content in normal exosomes used as the standard, and the miRNA is at least one selected from the group comprising mmu-mir-139-3p, mmu-mir-6240, mmu-mir-6412, mmu-mir-6907-5p_mmu-7019-5p, mmu-mir-7042-5p, and mmu-mir-7668-3p,
[7] the composition for activating the skin barrier of the item [4] or [5], which is a food composition or a cosmetic composition,
[8] a method for activating the skin barrier, comprising a step of administering an effective amount of β-alanine or a salt thereof to a subject,
[9] a method for activating the skin barrier, comprising the step of administering an effective amount of a culture product secreted from muscle cells by β-alanine or a salt thereof to a subject,
[10] the method for activating the skin barrier of the item [9], wherein the culture product is an exosome, and
[11] a method for producing exosomes, comprising the steps of treating muscle cells with β-alanine or a salt thereof, and recovering exosomes from the treated muscle cells.

### Advantageous Effects of Invention

The composition for activating the skin barrier of the present invention can improve the skin barrier of the skin.

### BRIEF DESCRIPTION OF THE DRAWINGS

FIG. 1 is a graph showing the activation of keratin 10 (A), involucrin (B), or filaggrin (C) genes in HaCaT cells by supernatants of C2C12 cells supplemented with β-alanine.

FIG. 2 is a graph showing the activation of keratin 10 (A), involucrin (B), or filaggrin (C) genes in HaCaT cells by exosomes from C2C12 cells supplemented with β-alanine.

FIG. 3 is a graph showing the activation of keratin 10 (A) or involucrin (B) genes in UVB-treated HaCaT cells by exosomes from C2C12 cells supplemented with β-alanine.

FIG. 4 is a graph showing the increased miRNA contained in exosomes secreted from C2C12 cells by β-alanine.

### DESCRIPTION OF EMBODIMENTS

### [1] Composition for Activating Skin Barrier

The composition for activating skin barrier of the present invention contains β-alanine or a salt thereof.

β-alanine is a compound represented by the following formula [1]:

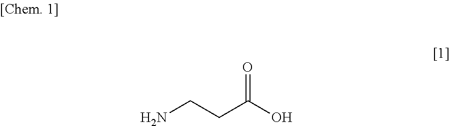

and is also called 3-aminopropanoic acid.

Extracts, concentrates, or purified products from foods or natural products that contain relatively large amounts of β-alanine, can be used, as β-alanine comprised in the composition for activating skin barrier of the present invention. Further, synthetic β-alanine may also be used. For example, β-alanine can be synthesized from β-propiolactone by a method for synthesizing β-alanine (Ford, Org. Sys. Coll. Vol. 3, 34 (1955)). As another synthesis method, it can be synthesized from acrylonitrile and ammonia. The composition for activating skin barrier of the present invention can also contain β-alanine as its salt, hydrate, or solvate.

The salt of β-alanine is not limited, so long as it is a salt with an inorganic base or an organic base, or a salt with an acid, and is a salt acceptable for medicine food or cosmetic. Specific examples of the salt with the inorganic base or the organic base include a salt with an inorganic base, an organic base, or a metallic alkoxide. They can be prepared by mixing β-alanine or glycine with an inorganic base, an organic base, or a metallic alkoxide.

As the inorganic bases that can form salts, there may be mentioned a hydroxide, carbonate, hydrogen carbonate, acetate, or hydride of alkali metals (such as lithium, sodium, potassium, or the like); a hydroxide, hydride, or the like of alkaline earth metals (such as magnesium, calcium, or barium). As the organic bases that can form salts, there may be mentioned dimethylamine, triethylamine, piperazine, pyrrolidine, piperidine, 2-phenylethylamine, benzylamine, ethanolamine, diethanolamine, pyridine, collagen, or the like. Further, as the metallic alkoxide, there may be mentioned sodium methoxide, potassium tert-butoxide, magnesium methoxide, or the like. The salt of β-alanine is preferably a sodium salt, potassium salt, calcium salt, or a combination thereof.

Specific examples of the salt with an acid include a salt with an inorganic acid or an organic acid. As the inorganic acid that can form a salt, there may be mentioned hydrochloric acid.

### <<Skin Barrier>>

The skin is composed of three layers: the epidermis, dermis, and subcutaneous tissue. The epidermis is mainly composed of cells called keratinocytes, and is 0.06-0.2 mm thick.

The dermis is elastic, contains blood vessels, nerves, and lymphatic vessels, and is 2.0-2.2 mm thick. It also contains mast cells and histiocytes. The dermis is mostly composed of a fibrous protein called collagen. A gel-like matrix such as hyaluronic acid fills the gaps while retaining moisture. In addition, a fibrous protein called elastin is added, and whereby giving the skin elasticity. The cells that produce these fibers and matrix are called fibroblasts. The subcutaneous tissue is a connective tissue with low fiber density that connects the skin to the underlying tissues such as fascia.

The skin barrier is also called the external barrier, and is mainly provided by the epidermis. The epidermis is composed of the stratum corneum, stratum granulosum, stratum spinosum, and stratum basale, and the stratum corneum contains sebum, intercellular lipids (ceramides, etc.), and natural moisturizing factors (amino acids, salts, etc.). The skin barrier plays a role in preventing the invasion of external stimuli and foreign substances (allergy-causing substances, bacteria, and viruses).

### <<Filaggrin>>

Filaggrin is present in the stratum corneum of the skin. Reduction or disappearance of filaggrin causes a decrease in barrier function. For example, filaggrin acts as a barrier function in atopic dermatitis.

### <<Involucrin>>

Involucrin is present in the stratum corneum of the skin. Involucrin forms a cross-linked structure with multiple proteins to form a cornified envelope (CE), which acts on the barrier function of the stratum corneum.

### <<Keratin 10>>

Keratin 10 is present in the spinous and granular layers of the skin. Keratin 10 is associated with skin turnover. Skin turnover is related to maintaining the functionality of the skin and acts on the barrier function.

### <<Activation of Skin Barrier Function Via Muscle Cells>>

The composition for activating the skin barrier of the present invention can directly act on cells to activate the skin barrier. On the other hand, the composition for activating the skin barrier of the present invention can act on muscle cells in vivo or in vitro and activate the skin barrier of cells via a culture (component) product secreted from the muscle cells, although this is not limited thereto. Therefore, the composition for activating the skin barrier of the present invention may contain a culture product secreted from muscle cells by β-alanine or a salt thereof. The culture product contains, but is not limited to, myokines (IL-15, etc.) secreted from muscle cells, and these myokines can activate the skin barrier of the skin.

As used herein, the term “culture product” includes culture supernatants and cultured cells obtained by contacting muscle cells with β-alanine or a salt thereof. Muscle cells include cultured cells or muscle cells in the living body. The cultured cells is not limited, but there may be mentioned C2C12 cells, primary skeletal muscle cultured cells, HSkMC cells (skeletal muscle cells), HSKMM cells (skeletal muscle myoblasts), or ioSkeletal Myocytes (skeletal muscle cells derived from human iPS cells). The muscle cells may be human muscle cells, or may be mammalian or avian muscle cells as described below.

The composition for activating the skin barrier of the present invention may contain a culture (substance) secreted from muscle cells by β-alanine or a salt thereof (hereinafter, sometimes referred to as β-alanine, or the like). The culture product (substance) secreted from muscle cells by β-alanine or the like. is not particularly limited as long as it has an skin barrier activation effect, but for example includes an exosome. Therefore, the composition for activating the skin barrier of the present invention may comprise exosomes secreted from muscle cells by β-alanine or the like.

The exosomes are membrane-bound extracellular vesicles (EVs) formed in the endosomal compartment of eukaryotic cells. There is no limit to the size of exosomes, but they have a diameter of about 30-150 nm, with most being 100 nm or less. Exosomes contain proteins or RNA, and can be secreted outside the cell and taken up by other cells.

Exosomes include, but are not limited to, miRNA. miRNA binds to mRNA to destabilize it and inhibit protein synthesis, thereby playing an important role in biological control.

The exosomes used in the present invention have a different content of mmu-mir-139-3p, mmu-mir-6240, mmu-mir-6412, mmu-mir-6907-5p_mmu-7019-5p, mmu-mir-7042-5p, or mmu-mir-7668-3p compared to normal exosomes. Specifically, but not limited to, one or more miRNAs, mmu-mir-139-3p, mmu-mir-6240, mmu-mir-6412, mmu-mir-6907-5p_mmu-7019-5p, mmu-mir-7042-5p, or mmu-mir-7668-3p, are increased.

The miRNA is a mouse miRNA. Mouse miRNA and human miRNA are orthologous, and the functions of many genes are conserved.

The composition of the present invention may be a composition that contains β-alanine or a salt thereof and causes muscle cells to secrete exosomes having an skin barrier activation effect.

The formulation of the composition for activating the skin barrier of the present invention may be: is not particularly limited, for example, there may be mentioned oral agents, such as powders, subtle granules, granules, tablets, capsules, suspensions, emulsions, sylups, extracts, or balls; or parenteral agents, such as injections, liquid for external use, ointments, suppositorys, creams for local administration, or eye-drops.

The above oral agent can be prepared in accordance with conventional methods, using excipients, such as gelatin, alginate sodium, starch, cornstarch, saccharose, lactose, glucose, mannitol, carboxymethyl-cellulose, dextrin, polyvinyl pyrrolidone, clystalline cellulose, soy lecithin, sucrose, fatty acid ester, talc, magnesium stearate, polyethylene glycol, magnesium silicate, silicic anhydride, or synthetic aluminum silicate; binders, disintegrators, surfactants, lubricants, flow accelerator, diluents, preservatives, colorants, flavors, correctives, stabilizers, humectants, antiseptics, antioxidant, or the like.

Examples of the parenteral agents include injections. In a preparation of the injections, an aqueous solvent such as normal saline solution or Ringer solution, non-aqueous solutions such as plant oil or fatty acid ester, a tonicity agent such as glucose or sodium chloride, a solubility assisting agent, a stabilizing agent, an antiseptic agent, a suspending agent, or an emulsifying agent, may be optionally used, in addition to the active ingredient.

The dosage when using the composition for activating the skin barrier can be appropriately determined depending on, for example, the age, sex, weight, or administration method of the subject, and it can be administered orally or parenterally. For example, the dosage when the composition for activating the skin barrier of the present invention is orally ingested is preferably 0.01 to 100 mg/kg per day as β-alanine for an adult, in one embodiment 0.05 to 50 mg/kg, and in another embodiment 0.1 to 10 mg/kg. Note that the above administration method is one example, and other administration methods may also be used. It is desirable to determine the administration method, dosage, administration period, administration interval, etc. of the composition for activating the skin barrier to humans through a controlled clinical trial.

Furthermore, the dosage form is not limited to pharmaceuticals, and it is possible to administer the composition as a food composition (e.g., functional food, health food, beverage), cosmetic composition, or animal feed composition, as described below.

The composition for activating the skin barrier containing β-alanine can be produced using a known method for producing a pharmaceutical composition, food composition, cosmetic composition, or animal feed composition, except that it contains β-alanine as an active ingredient. In addition, the composition can be produced using a method for producing a known pharmaceutical composition, food composition, cosmetic composition, or animal feed composition, except that it contains a culture product (substance; e.g., exosome) secreted from muscle cells by β-alanine or the like as an active ingredient.

The composition for activating the skin barrier of the present invention may contain other components. Examples of the other components include, for example, emulsifiers such as edible fats and oils, water, glycerin fatty acid ester, sucrose fatty acid ester, sorbitan fatty acid ester, propylene glycol fatty acid ester, glycerin organic acid fatty acid ester, polyglyceryl fatty acid ester, calcium stearoyl lactylate, sodium stearoyl lactate, polyoxyethylene sorbitan fatty acid ester; thickening stabilizers such as locust bean gum, carrageenan, alginic acids, pectin, xanthan gum, crystalline cellulose, carboxymethyl cellulose, methyl cellulose, agar, glucomannan, gelatin, starch, or chemical starch; salty taste agents such as salt, or potassium chloride; acidulants such as acetic acid, lactic acid, or gluconic acid; sugars or sugar alcohols; sweeteners such as stevia or aspartame; colorants such as beta-carotene, caramel, or red koji pigment; antioxidants such as tocopherol or tea extract; food materials or food additives such as flavoring agent; pH adjuster; food preservative, or shelf life improver. Further, the composition for activating the skin barrier may contain various vitamins, or functional materials such as coenzyme Q, plant sterol, or milk fat globule membrane. The amount of these other components is preferably 80% by mass or less, more preferably 40% by mass or less, and further preferably 20% by mass or less, as a total amount in the composition for activating the skin barrier of the present invention.

### <<Food Composition>>

The composition for activating the skin barrier of the present invention may be a food composition. The food composition for activating the skin barrier of the present invention contains β-alanine or a salt thereof. The food composition for activating the skin barrier of the present invention may also contain a culture product (substance; for example, exosome) produced from muscle cells by β-alanine or the like. The food composition for activating the skin barrier of the present invention is not particularly limited as long as it can be administered orally.

The food in the food composition for activating the skin barrier of the present invention is a food or drink, and includes a beverage. The food in the present invention is not particularly limited, for example, there may be mentioned seasonings such as a bean paste, soy sauce, sauce for noodles, sauce, soup, pasta sauce, dressing, mayonnaise, tomato ketchup, Worcestershire sauce, sauce for pork cutlet, or sprinkle; instant cooked foods such as a soup base, curry roux, white sauce, rice with tea base, or soup base; soups such as miso soup, soup, consomme soup, or potage soup; processed livestock products such as grilled meat, ham, or sausage; processed marine products such as boiled fish paste, dried fish, salted fish guts, fish boiled in soy sauce, rare delicacy; processed vegetable products such as pickles; snacks such as potato chips, or rice cracker; bakery foods such as bread, sweet bread, or cookies; cooked foods such as boiled foods, fried foods, grilled foods, curry, stew, gratin, rice, porridge, or rice ball; noodles such as pasta, wheat noodle, or ramen; oil processed foods such as margarine, shortening, fat spread, or flavored fat spread; materials for confectionery and bread such as flower pastes, or bean paste; mixed powders such as bread mix powder, cake mix powder, or fried food mix powder; confectioneries such as chocolate, candy, jelly, ice cream, or gum; Japanese confectioneries such as steamed bun, or castella; beverages such as coffee, coffee milk, tea, milk tea, soy milk, nutritional drink, vegetable drink, vinegared drink, juice, cola, mineral water, or sports drink; alcoholic beverages such as beer, wine, cocktail, or sour; milk and dairy products such as bovine milk, yogurt, or cheese.

The food composition for activating the skin barrier of the present invention can be produced using the known methods for producing foods and beverages, except that it contains β-alanine, etc., or a culture product (substance; for example, exosomes) produced from muscle cells by β-alanine, etc.

### <<Cosmetic Composition>>

The composition for activating the skin barrier of the present invention may be a cosmetic composition. The cosmetic compositions for activating the skin barrier of the present invention comprises β-alanine or a salt thereof. It can activate the skin barrier by comprising β-alanine and acting on epidermal cells. In addition, the cosmetic compositions for activating the skin barrier of the present invention also contain culture product (substance; e.g., exosome) produced from muscle cells by β-alanine or the like. As the specifical cosmetics, there may be mentioned serums, cosmetic liquid, cleansing, emulsions, creams, lipsticks, foundations, gels, packs, white powders, blushes, hair tonics, shampoos, rinses, sunscreens, facial cleansers, or lip balms.

The amount of β-alanine or the like in the cosmetic composition of the present invention is not particularly limited as long as the effect of the present invention can be achieved, but is, for example, 0.1 to 100% by weight, preferably 1 to 50% by weight, and more preferably 1 to 25% by weight. The amount of culture product (substance; e.g., exosome) produced from muscle cells by β-alanine or the like in the cosmetic composition of the present invention is not particularly limited as long as the effect of the present invention can be achieved, but is, for example, 0.1 to 100% by weight, preferably 1 to 50% by weight, and more preferably 1 to 25% by weight.

The cosmetic composition of the present invention, as long as it does not inhibit the effects of the present invention, may contain moisturizing agents (e.g., trimethylglycine, N-[2-hydroxy-3-(trimethylammonio) propyl] hydrolyzed wheat protein chloride, hyaluronic acid, sodium pyrrolidone carboxylic acid, betaine, jojoba oil, hydrolyzed keratin), colorants (e.g., pigment, or dye), viscosity modifiers (e.g., methylcellulose), emulsifying agents (e.g., glycerol monostearate), pearlescent agents (e.g., glycol distearate, or ethylene glycol distearate), salts (e.g., sodium chloride), plant extracts, preservatives (e.g. methylparaben, propylparaben, butylparaben, 1,3-butylene glycol (1,3-butanediol), phenoxyethanol, or pentylene glycol (1,2-pentanediol)), vitamins, fragrances, UV absorbers, antioxidants, wetting agents, chelating agent, pH adjuster (e.g. citric acid, or tartaric acid), and water.

### <<Animal Feed Composition>>

The composition for activating the skin barrier of the present invention can be used as an animal feed composition. Examples of the feed include, but are not limited to, feed for industrial animals and feed for pets (pet food).

### [2] Method for Activating the Skin Barrier

The method for activating the skin barrier of the present invention comprises a step of administering an effective amount of β-alanine or a salt thereof to a subject. The method for activating the skin barrier of the present invention may be performed when the subject is healthy or when the subject is suffering from a disease. That is, the method may be performed as a medical practice or a non-medical practice. When the subject is an animal, the method may be performed as a feeding method. The skin barrier can be activated by administering an effective amount of the composition for skin barrier activation to a human or an animal.

The method for activating the skin barrier of the present invention comprises a step of administering an effective amount of a culture product secreted from muscle cells by β-alanine or a salt thereof to the subject. Examples of the culture product include, but are not limited to, exosomes. The method for activating the skin barrier of the present invention may be performed when the subject is healthy or when the subject is suffering from a disease. That is, the method may be performed as a medical practice or a non-medical practice. The culture product (particularly exosomes) can be used in the method for activating the skin barrier.

The culture product of the present invention can be produced by contacting β-alanine or a salt thereof with muscle cells. Active ingredients in the culture product include exosomes, but there are also active ingredients other than exosomes, which have not yet been identified at this time. Therefore, this specification discloses a culture product that produces β-alanine or a salt thereof by contacting muscle cells.

### <<β-Alanine Used in Method for Activating the Skin Barrier>>

Said β-alanine can be used in a method for activating the skin barrier. That is, this specification discloses β-alanine used in a method for activating the skin barrier.

The β-alanine can also be used to promote filaggrin, involucrin, or keratin 10.

### <<Use of β-Alanine in the Manufacture of a Composition for Skin Barrier Activation>>

Said β-alanine, etc. (culture, particularly exosomes) can be used in the manufacture of a composition for skin barrier activation. That is, this specification discloses the use of β-alanine, etc. in the manufacture of a composition for skin barrier activation.

### [Functions]

The mechanism by which the composition for activating the skin barrier of the present invention can activate the skin barrier has not been elucidated in detail, but can be presumed as follows.

The epidermis of the skin is composed of the stratum corneum, stratum granulosum, stratum spinosum, and stratum basale. In the composition for activating the skin barrier of the present invention, β-alanine acts directly on the epidermis to promote the expression of filaggrin, involucrin, or keratin 10, and activate the skin barrier function. In addition, it is considered that β-alanine acts on muscle cells to produce substances (e.g., exosomes) that are effective in activating the skin barrier function from the muscle cells. Therefore, it is presumed that the skin barrier function can be activated via substances (e.g., exosomes) that are effective in activating the skin barrier function produced by muscle cells.

### [3] A Method for Preparing Exosome

The method for preparing exosome of the present invention comprises a step of treating muscle cells with β-alanine or a salt thereof, and a step of recovering exosomes from the treated muscle cells. In the method for preparing exosome of the present invention, the muscle cells described in the above section “[1] Composition for activating skin barrier” can be used. The origin of the muscle cells is not particularly limited, but may be muscle cells derived from human, for example, muscle cells derived from mammalian or avian. Specific examples include horses, pigs, poultry (e.g., turkeys, ducks, chickens, broilers, layers), dogs, cats, rabbits, hamsters, guinea pigs, and pets such as squirrels; mice, rats, cows, goats, sheep, giraffes, bison, yaks, buffalo, deer, camels, alpacas, llamas, and antelopes. The prepared exosomes are the exosomes described in the above section “[1] Composition for activating skin barrier”.

The concentration of β-alanine in the step of treating muscle cells with β-alanine or a salt thereof is not particularly limited as long as the effects of the present invention are obtained, but the lower limit is, for example, 0.001 mM or more, 0.01 mM or more in one embodiment, 0.05 mM or more in one embodiment 0.1 mM or more in one embodiment. The upper limit is, for example, 1000 mM or less, 100 mM or less in one embodiment 10 mM or less in one embodiment 5 mM or less in one embodiment. The upper and lower limits can be appropriately combined to create a suitable range.

The method for preparing exosome of the present invention can also treat muscle cells in vivo with β-alanine or a salt thereof. When preparing exosomes from muscle cells in vivo, β-alanine or a salt thereof can be administered to the living body, and the dosage can be appropriately determined by a person skilled in the art, but is, for example, 0.01 to 100 mg/kg of β-alanine per day, preferably 0.05 to 50 mg/kg, and preferably 0.1 to 10 mg/kg, and the upper and lower limits can be appropriately combined to create a suitable range.

### EXAMPLES

The present invention will now be further illustrated by, but is by no means limited to, the following Examples.

### Example 1

In this example, muscle cells were treated with β-alanine, the cell supernatant was added to HaCaT cells, and the effect on the expression of filaggrin, involculin, or keratin 10 was examined.

C2C12 cells were seeded in a 6-well plate at a concentration of 2.0×10⁵ cells/mL, and after 48 hours, the medium was replaced with DMEM medium containing 2% Horse Serum (HS) (Thermo Fisher Scientific) to induce differentiation.

After a further 24 hours, the medium was replaced with DMEM medium containing 2% HS. The medium was then replaced every two days, and on the 8th and 9th days after cell seeding, the medium was replaced and β-alanine was added (0.5 mM or 1 mM). In addition, 1×PBS was added in an amount equal to the amount of β-alanine added as a control.

The culture supernatant on the 10th day after seeding was used in the experiment. HaCaT cells were seeded in a 6-well plate at a concentration of 3.0×10⁴ cells/mL and cultured for 24 hours. The culture supernatant was then removed, and the collected C2C12 cell culture supernatant was added at 2.0 mL/dish. FBS was added at 176 μL/dish. After a further 24 hours of culture, the supernatant was added in the same manner. FBS was added so that the FBS concentration in the culture medium was 10% of the culture medium, including the HS contained in the C2C12 cell culture supernatant. Total RNA was prepared 72 hours after seeding.

The medium was removed by aspiration, and 1×PBS was added at 1 mL/well to wash the cells.

Washing was performed twice. After that, 1×PBS (200 μL/well) and lysis/-binding buffer (400 μL/well) were added and spread throughout the dish, and the entire amount was collected in a 1.5 mL tube. The collected sample was suspended for 60 seconds using a vortex mixer. The filter tube and collection tube were assembled, and the sample solution was added to the filter tube, followed by centrifugation at 4° C. and 10,000 rpm for 15 seconds. The liquid discharged into the collection tube was discarded, and the filter tube and collection tube were reassembled. 90 μL of DNase Incubation Buffer per sample and 10 μL of DNase I per sample were mixed in a 1.5 mL tube. This mixture was added to the filter tube and incubated at room temperature for 15 minutes. After incubation, Wash buffer I (500 μL) was added to the filter tube and centrifuged at 10,000 rpm at 4° C. for 15 seconds. The liquid discharged into the recovery tube was discarded, and the filter tube and recovery tube were reassembled. Wash buffer II (500 μL) was added to the filter tube and centrifuged at 10,000 rpm at 4° C. for 15 seconds. The liquid discharged into the recovery tube was discarded, and the filter tube and recovery tube were reassembled. Wash buffer II (200 μL) was added to the filter tube and centrifuged at 13,000×g at 4° C. for 2 minutes. The filter tube was inserted into a new 1.5 mL tube, and Elution buffer (70 μL) was added to the filter tube. After standing at room temperature for 3 minutes, it was centrifuged at 10,000 rpm at 4° C. for 1 minute. The eluate obtained by the above procedures was used as an RNA solution. The RNA concentration in the solution was calculated based on the absorbance value at 260 nm using a NanoDrop 2000/2000c spectrophotometer (Thermo Fisher Scientific) and was used in subsequent experiments.

cDNA was synthesized as follows: 5 pmol of Oligo (dT) 20 primer (TOYOBO, Osaka, Japan) was added to 1.0 μg of total RNA extracted from cells, and RNase-free water was added to bring the total volume to 13 μL. The heat treatment reaction was carried out at 65° C. for 5 minutes using a GeneExplorer Thermal Cycler (Hangzhou Bioer Technology Co., Ltd. Zhejiang, China), then immediately transferred to ice for rapid cooling and left to stand for 5 minutes. The reverse transcriptase reaction program in the Thermal Cycler was advanced to the 37° C. stage and paused. A mixed solution of 4 μL of reverse transcriptase reaction buffer RT buffer (TRT) (TOYOBO), 2 μL of 10 mM dNTPs (GE Healthcare, Chicago, IL), and 0.5 μL of reverse transcriptase Rever TraAce (100 units/μL) (TOYOBO) was added per sample and mixed gently. The sample was spun down and placed back in the Thermal Cycler, and reacted at 37° C. for 15 minutes, 50° C. for 5 minutes, and 99° C. for 5 minutes to synthesize cDNA. This cDNA was used as a template for quantitative RT-PCR.

Quantitative RT-PCR was performed using the prepared cDNA as a template. 49 μL of sterile water, 3.5 μL each of forward and reverse primers diluted to 10 μM, 7.0 μL of template cDNA (cDNA was diluted 10-fold for the target gene and 50-fold for β-actin), and 24.5 μL of THUNDERBIRD SYBR qPCR Mix (TOYOBO) were placed in a 0.2 mL tube and thoroughly suspended on ice. 25 μL of each mixed solution was added to 3 wells of a 96-well PCR plate (NIPPON Genetics), and quantitative RT-PCR was performed using a Thermal Cycler Dice Real Time System (Takara Bio). The PCR reaction consisted of an initial denaturation at 95° C. for 30 seconds, followed by 45 cycles of three steps: 95° C. for 5 seconds (denaturation), 60° C. for 10 seconds (annealing), and 72° C. for 20 seconds (extension reaction). The expression levels of the target genes detected by FAM were relatively quantified using the ΔΔCt method. Primer synthesis was outsourced to FASMAC Corporation (Kanagawa, Japan). The sequences of the primers used are as follows:

K10 forward:
(SEQ ID NO: 1)
5′-TGATGTGAATGTGGAAATGAATGC-3′
K10 reverse:
(SEQ ID NO: 2)
5′-GTAGTCAGTTCCTTGCTCTTTTCA-3′
Involucrin forward:
(SEQ ID NO: 3)
5′-GGGTGGTTATTTATGTTTGGGTGG-3′
Involucrin reverse:
(SEQ ID NO: 4)
5′-GCCAGGTCCAAGACATTCAAC-3′
Filaggrin forward:
(SEQ ID NO: 5)
5′-GGGCACTGAAAGGCAAAAAG-3′
Filaggrin reverse:
(SEQ ID NO: 6)
5′-CACCATAATCATAATCTCCACTACCA-3′

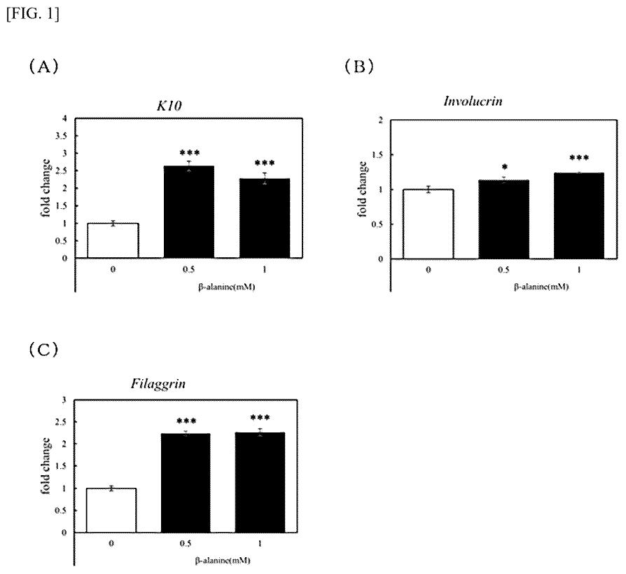

As shown in FIG. 1, the supernatant of C2C12 cells by β-alanine increased the expression mRNA of filaggrin, involucrin and keratin 10 in HaCaT cells.

### Example 2

In this example, muscle cells were treated with β-alanine, and exosomes were collected from the cell supernatant. The collected exosomes were added to HaCaT cells, and the effects on the expression of filaggrin, involucrin, and keratin 10 were examined.

C2C12 cells were seeded in a 10 mL dish at 2× 10⁵ cells/mL. The cells were cultured in DMEM medium containing 10% Exosome-depleted FBS Media Supplement Heat inactivated. After 48 hours, the medium was replaced with DMEM medium containing 2% serum Exosome-depleted FBS Media Supplement Heat Inactivated, and differentiation was induced. After another 24 hours, the medium was replaced with DMEM medium containing 2% Exosome-depleted FBS Media Supplement Heat Inactivated. Thereafter, the medium was replaced every 2 days, and the medium was replaced, and β-alanine was added on the 8th and 9th days after cell seeding. For β-alanine, β-alanine prepared at 100 mM in a DMEM medium containing 2% Exosome-depleted FBS Media Supplement Heat Inactivated was used, and the medium was removed by the amount of β-alanine added. Exosomes were purified from the supernatant on the 10th day after seeding. The control supernatant was the untreated supernatant in which β-alanine was not added but only the medium was exchanged.

For each sample, the supernatant of two 10-mL Dish sheets was collected. 10 mL of the collected culture supernatant was centrifuged at 300×g for 5 minutes to remove cells from the culture supernatant. The supernatant was transferred to another tube and then centrifuged at 1,200×g for 20 minutes to remove cell fragments. The supernatant was transferred to another tube and centrifuged at 10,000×g for 30 minutes to remove extracellular vesicles larger than exosomes. The culture supernatant, from which cells and large-sized extracellular vesicles had been removed, was concentrated approximately 40-fold using a centrifugal ultrafiltration unit (AmiconUltra-15 100K, Merck Millipore) with a filter with a molecular weight cutoff of 100,000. Exosomes were purified from the concentrated culture supernatant using the MagCapture Exosome Isolation PS Kit Ver. 2 (FUJIFILM Wako).

First, a buffer was prepared. 0.55 mL of Exosome Immobilizing/Washing Buffer (10×) and 4.95 mL of purified water were added to a 15-mL tube, and then Exosome Immobilizing/Washing buffer (1×) was prepared by adding 11 μL of Exosome Binding Enhancer (500×). Exosome Elution Buffer (1×) was prepared by adding 15 μL of Exosome Elution Buffer (10×) and 135 μL of purified water to a 1.5-mL tube. To the Exosome Elution Buffer, EV-Save™ Extracellular Vesocle Biocking Reagent (FUJIFILM Wako) was added as an exosome protection agent at a dilution of 1/100.

Next, Exosome Capture immobilized beads were prepared. Sixty microliters of Biotin Capture Magnetic Beads which is agitated well by a vortex mixer, were placed in a 1.5-mL reaction tube, and 500 μL of Exosome Immobilizing/Washing buffer (1×) was added to the tube, and the mixture was suspended by a vortex mixer. Then, the tube was spun down and set on a magnetic stand, and allowed to stand for about 1 minute. Next, 500 μL of Exosome Immobilizing/Washing buffer (1×) and 10 μL of Biotin-labeled Exosome Capture were added to the tube, and the tube was removed from the magnetic stand, and suspended by a vortex mixer. Then, the tube was reacted for 10 minutes by inverting and mixing by a rotating stirrer at room temperature. The tube was spun down, set on the magnetic stand again, and allowed to stand for about 1 minute. When the magnetic beads completely adhered to the tube wall, the supernatant was removed with a pipette. (a) 500 μL of Exosome Immobilizing/Washing buffer (1×) was added to the tube, and the tube was removed from the magnetic stand, suspended by a vortex mixer, spun down, set on the magnetic stand again, and allowed to stand for 1 minute. When the magnetic beads completely adhered to the tube wall, the supernatant was removed with a pipette. The above procedure (a) was repeated one more time. The Exosome Capture immobilized beads were completed by the above procedure.

Next, the Exosome Capture immobilized beads were reacted with the culture supernatant concentrated as described above. About 500 μL of the 40-fold concentrated culture supernatant was transferred to a sterile 1.5-mL tube, and 1/500 volume of Exosome Binding Enhancer (500×) was added to the cell supernatant and mixed using a vortex mixer. The tube was spun down and the sample was transferred to a tube containing Exosome Capture immobilized beads (Reaction Tube) and mixed by a vortex mixer. The reaction was carried out at room temperature for at least 1 hour while mixing invertedly with a rotary stirrer. After spinning down the 1.5-mL Reaction tube, it was set on the magnetic stand and allowed to stand for about 1 minute. When the magnetic beads completely adhered to the tube wall, the supernatant was removed with a pipette and the exosomes were bound to the beads.

Next, the exosome-binding beads were washed. (b) Add 1 mL of Washing Buffer containing Exosome Binding Enhancer to a 1.5 mL Reaction tube containing exosome-binding beads, and the mixture was suspended by a vortex mixer. The 1.5 mL Reaction tube was spun down and set on a magnetic stand, and allowed to stand for about 1 minute.

When the magnetic beads completely adhered to the tube wall, the supernatant was removed.

Further, the above procedure (b) was repeated twice. The washed exosome-bound beads were prepared by this procedure.

Exosomes were eluted by the following procedure. 50 μL of Exosome Elution Buffer (1×) was added to a 1.5 mL Reaction tube containing washed exosome-bound beads, and then the Reaction tube was removed from the magnetic stand, and suspended by a vortex mixer. The tube was spun down, set on the magnetic stand, and allowed to stand for 1 minute. After the magnetic beads had completely adhered to the tube wall, the supernatant was collected into a new sterile 1.5-mL tube. Further, 50 μL of Exosome Elution Buffer (1×) was added to the magnetic beads remaining in the 1.5-mL reaction tube, and the tube was removed from the magnetic stand, and suspend by a vortex mixer. The tube was spun down, set on the magnetic stand, and allowed to stand for 1 minute. When the magnetic beads completely adhered to the tube wall, the supernatant was collected in a new sterile 1.5-mL tube (as described above), to be a total of 100 μL of exosome solution.

HaCaT cells were seeded in a 5 mL dish at a concentration of 3.0×10⁴ cells/mL and cultured for 24 hours. The culture supernatant was removed, and purified exosomes were added at 1 μg/well and cultured for 48 hours.

Total RNA was collected and cDNA was synthesized in the same manner as in Example 1.

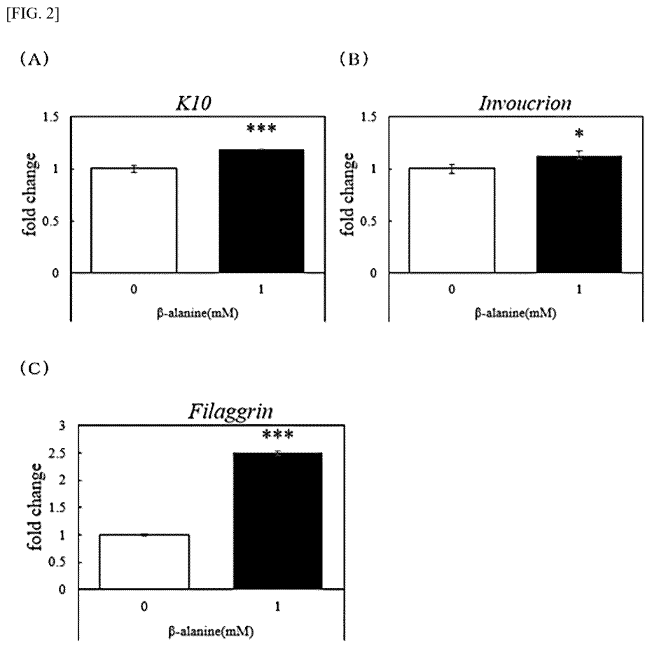

As shown in FIG. 2, exosomes derived from differentiated C2C12 cells treated with 1 mM β-alanine showed enhanced expression of filaggrin, involucrin, and keratin 10.

### Example 3

In this example, muscle cells were treated with β-alanine, and exosomes were collected from the cell supernatant.

The collected exosomes were added to UVB-treated HaCaT cells, and the effects on the expression of involucrin and keratin 10 were examined.

HaCaT cells were irradiated with 10 mJ/cm2 UVB and seeded. Four hours after seeding, 1 μg/well of exosomes derived from β-alanine-treated differentiated C2C12 cells obtained in Example 2 were added, and 24 hours after addition, the expression levels of keratin 10 (K10: A) and involucrin (Involucrin: B) were measured by RT-qPCR. As a control, HaCaT cells to which untreated exosomes derived from differentiated C2C12 cells were added after UVB irradiation were used. In addition, the change in expression of skin barrier-related genes due to UVB irradiation was confirmed using HaCaT cells to which exosomes derived from differentiated C2C12 cells that had not been irradiated or treated with UVB had been added.

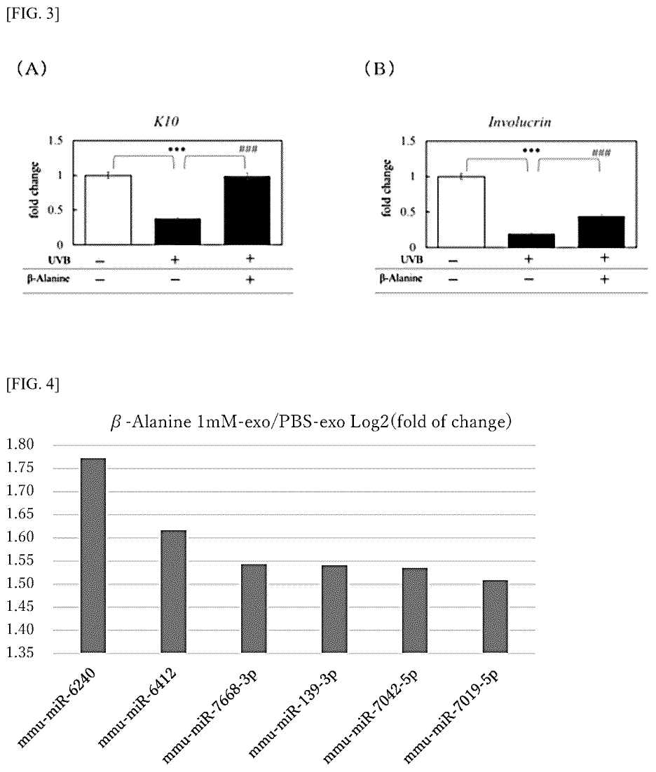

As shown in FIG. 3, the expression levels of keratin 10 and involucrin, which had decreased due to UVB stimulation, were restored by exosomes derived from differentiated C2C12 cells treated with β-alanine.

In this example, miRNAs in exosomes were analyzed by microarray.

The exosome solution purified in Example 2 was used for microarray analysis. The microarray experiment was outsourced to Kamakura Techno Science (Kanagawa, Japan). Based on the data obtained, samples with a ratio of 1.5 or more compared to the control and one of the signal values of two samples exceeding 100 were extracted as miRNAs with altered transcription levels.

As a result of the analysis, six RNAs were increased by β-alanine treatment, as shown in Table 1.

TABLE 1
Increased miRNA

mmu-mir-139-3p
mmu-mir-6240
mmu-mir-6412
mmu-mir-6907-5p_mmu-7019-5p
mmu-mir-7042-5p
mmu-mir-7668-3p

Furthermore, as shown in FIG. 4, six miRNAs were increased, and 14 miRNAs were decreased.

### INDUSTRIAL APPLICABILITY

The composition for activating the skin barrier of the present invention can improve the skin barrier function of the epidermis of the skin.

### [Sequence Table]

Patent Application YGK24002P2024-046041_1.xml

1. A composition for activating skin barrier, comprising β-alanine or a salt thereof.

2. The composition for activating the skin barrier according to claim 1, wherein filaggrin, involucrin, or keratin 10 is activated.

3. A composition for activating the skin barrier, comprising a culture product secreted from muscle cells by β-alanine or a salt thereof.

4. The composition for activating the skin barrier according to claim 3, wherein the culture product is an exosome.

5. A composition for secreting an exosome having a skin barrier activation effect from muscle cells, comprising-alanine or a salt thereof.

6. The composition according to claim 4, wherein miRNA content in the exosome varies with respect to miRNA content in normal exosomes used as the standard, and the miRNA is at least one selected from the group comprising mmu-mir-139-3p, mmu-mir-6240, mmu-mir-6412, mmu-mir-6907-5p_mmu-7019-5p, mmu-mir-7042-5p, and mmu-mir-7668-3p.

7. The composition for activating the skin barrier according to claim 4, which is a food composition or a cosmetic composition.

8. A method for activating the skin barrier, comprising a step of administering an effective amount of the composition of claim 1 to a subject.

9. A method for activating the skin barrier, comprising the step of administering an effective amount of the composition of claim 3 to a subject.

10. The method for activating the skin barrier according to claim 9, wherein the culture product is an exosome.

11. A method for preparing exosomes, comprising the steps of treating muscle cells with β-alanine or a salt thereof, and recovering exosomes from the treated muscle cells.

12. The composition according to claim 5, wherein miRNA content in the exosome varies with respect to miRNA content in normal exosomes used as the standard, and the miRNA is at least one selected from the group comprising mmu-mir-139-3p, mmu-mir-6240, mmu-mir-6412, mmu-mir-6907-5p_mmu-7019-5p, mmu-mir-7042-5p, and mmu-mir-7668-3p.

13. The composition for activating the skin barrier according to claim 5, which is a food composition or a cosmetic composition.


SEQUENCE LISTING (6 sequences)

SEQ ID NO: 1 - DNA, 24 bp, synthetic construct
  source 1..24: mol_type = other DNA
tgatgtgaatgtggaaatgaatgc

SEQ ID NO: 2 - DNA, 24 bp, synthetic construct
  source 1..24: mol_type = other DNA
gtagtcagttccttgctcttttca

SEQ ID NO: 3 - DNA, 24 bp, synthetic construct
  source 1..24: mol_type = other DNA
gggtggttatttatgtttgggtgg

SEQ ID NO: 4 - DNA, 21 bp, synthetic construct
  source 1..21: mol_type = other DNA
gccaggtccaagacattcaac

SEQ ID NO: 5 - DNA, 20 bp, synthetic construct
  source 1..20: mol_type = other DNA
gggcactgaaaggcaaaaag

SEQ ID NO: 6 - DNA, 26 bp, synthetic construct
  source 1..26: mol_type = other DNA
caccataatcataatctccactacca